(19) United States
(12) Patent Application Publication
(10) Pub. No.: US 2007/0276338 A1
(43) Pub. Date: Nov. 29, 2007

(54) Disposable syringe with built-in carpule

(76) Inventors: Ming-Jeng Shue, Taichung City (TW); Phillip Shue, Taichung City (TW); Deborah Huang, Taichung City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/601,526

(22) Filed: Nov. 16, 2006

(30) Foreign Application Priority Data
May 26, 2006 (TW) 095118782

(51) Int. Cl. A61M 5/00

(52) U.S. Cl. 604/187; 604/232

(57) ABSTRACT

A disposable syringe includes a barrel, a seat member held by a grip member in the barrel such that a needle cannula extends through the seat member, a carpule configured to be fitted in a rear compartment of the barrel, and having front and rear seal members to confine a sealed chamber communicated with the needle cannula for holding liquid medicament, and a plunger operable to move the rear seal member forwardly to expel the liquid medicament through the needle cannula so as to bring the carpule in a used-up position, and to further move the front seal member from the used-up position so as to remove the grip member from the seat member to thereby bring the seat member to the disengaging position. A biasing member is disposed to bias the seat member together with the carpule rearwardly so as to retract the used needle cannula into the barrel.

### CROSS-REFERENCE TO RELATED APPLICATION

This application claims priority of Taiwanese patent Application No. 095118782, filed on May 26, 2006.

### BACKGROUND OF THE INVENTION

1. Field of the Invention

This invention relates to a disposable syringe, more particularly to a disposable syringe with a carpule.

2. Description of the Related Art

In U.S. Pat. No. 6,221,055 B1 for a retractable dental syringe as shown in FIGS. 1 and 2, there is disclosed a pre-filled syringe 9, which includes a barrel 91 having an open widened rear end 911 to receive an operating end 973 of a plunger 97, a needle holder 93 for holding a double-ended needle 94, a push ring 92 for holding the needle holder 93 in the barrel 91, a biasing member 95 disposed between a front end 912 of the barrel 91 and the needle holder 93, and a carpule 96 positioned in the barrel 91 from the rear end 911. The carpule 96 has a front seal 963 disposed to seal a front end 962 thereof, and a slidable piston seal including an outer rim member 99 and a releasable core member 98 to seal a rear end 961 of the carpule 96 so as to define a sealed medicament chamber 964. The plunger 97 has inner and outer walls 971, 972 parallel to and spaced apart from each other and connected at the operating end 973.

In operation, the rear end 961 of the carpule 96 is associated with the front of the plunger 97. Then the plunger and carpule assembly is introduced into the barrel 91 from the rear end 911, and the carpule 96 is moved forwardly until the front seal 963 is punctured by a communicating end 941 of the needle 94, as shown in FIG. 1. As a tip end 942 of the needle 94 is inserted into a patient, the plunger 97 is pressed forwardly to inject the contents of the chamber 964 through the needle 94, as shown in FIG. 2. At the end of an injection, the plunger 97 is further pressed to move the carpule 96 forwardly to remove the push ring 92, thereby freeing the needle holder 93 for retraction of the needle holder 93, the needle 94, the front seal 963 and the core member 98 into the carpule 96 by virtue of a biasing action of the biasing member 95, as shown in FIG. 3.

Although the syringe 9 can be operated to retract the needle 94 for safe disposal, the following drawbacks arise:

1. The assembly of the carpule 96 and the plunger 97 to the barrel 91 before the injection operation is complicated and inconvenient to conduct. In addition, with such construction, the carpule 96 cannot be accommodated in the barrel 91 in advance, and the carpule 96 and the barrel 91 have to be packed separately for subsequent assembly in use, thereby resulting in increased operation time.

2. Since the used needle 94 and the needle holder 93 are retracted within the inner wall 971 of the plunger 97, before triggering the biasing action of the biasing member 95, the respective retaining engagements between the push ring 92 and the needle holder 93, between the front seal 963 and the carpule 96, and between the core member 98 and the outer rim member 99 have to be released at the same time. Thus, the retraction of the used needle 94 may fail. Such complicated structural relationship makes the manufacture of the syringe 9 troublesome.

### SUMMARY OF THE INVENTION

The object of the present invention is to provide a disposable syringe with a built-in carpule which ensures retraction of a used needle into a syringe barrel for safe disposal, which has a carpule that can be pre-assembled in the barrel before injection so that the injection operation is simplified and convenient to conduct, and which can be conveniently rendered unreusable after injection in a single operation.

According to this invention, the disposable syringe with a built-in carpule includes a barrel including front and rear barrel ends opposite to each other along an axis in an axial direction, a surrounding barrel wall which interconnects the front and rear barrel ends, and which includes front and rear wall portions that are disposed proximate to the front and rear barrel ends, respectively, and that define front and rear compartments, respectively. A needle cannula has a tip end which is disposed forwardly of the front barrel end in a position of use, and a communicating end which is opposite to the tip end along the axis. A seat member has a surrounding gripped portion surrounding the axis. A grip member is detachably retained on the rear wall portion, and is configured to be frictionally engaged with the gripped portion to provide a resisting force that guards the gripped portion against movement relative to the rear wall portion in the axial direction during a piercing action of the needle cannula for injection, and that permits disengagement of the grip member from the gripped portion so as to enable a subsequent movement of the seat member when the grip member is subjected to a pressing force. A carpule has a tubular wall which is configured to be fit in the rear compartment, and which extends towards the grip member to terminate at a tubular edge surface, and front and rear seal members which are spaced apart from each other in the axial direction, and which, in cooperation with the tubular wall, form a sealed chamber for holding liquid medicament. The rear seal member is disposed to be movable to abut against the front seal member in an initial course so as to expel the liquid medicament out of the sealed chamber, thereby placing the carpule in a used-up position. The front seal member has a mating surface which is configured to permit the communicating end to be retained with and to pass through the mating surface such that the needle cannula is in fluid communication with the sealed chamber, and such that once the rear seal member abuts against the front seal member in the used-up position, the pressing force is transmitted through the tubular wall to force the tubular edge surface against the grip member in a subsequent course so as to effect disengagement of the grip member from the gripped portion, thereby placing the seat member in a disengaging position, where the mating surface is closer to the front barrel end than when the carpule is in the used-up position. A plunger has a push end which is disposed to move the rear seal member forwardly along the tubular wall to bring the carpule to the used-up position, and to move the mating surface forwardly from the used-up position so as to permit the seat member to be placed in the disengaging position, and an operating end which is opposite to the push end in the axial direction, and which is disposed to extend outwardly of the rear barrel end to be subjected to an external force which is greater in the subsequent course than in the initial course. A biasing member is disposed in the front compartment such that, once the seat member is in the disengaging position, the biasing member biases the seat member together with the carpule towards the rear barrel end to retract the tip end of the needle cannula into the barrel, thereby placing the needle cannula in a disposal position.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a sectional view of a conventional retractable syringe in a ready-to-use position;

FIG. 2 is a sectional view of the conventional retractable syringe in a state after completion of an injection stroke;

FIG. 3 is a sectional view of the conventional retractable syringe, showing that a needle-holding structure is fully retracted into a carpule;

FIG. 4 is an exploded sectional view of the first preferred embodiment of a disposable syringe according to this invention;

FIG. 5 is a sectional view of the first preferred embodiment in an initial assembled position;

FIG. 6 is a fragmentary sectional view of the first preferred embodiment;

FIG. 7 is a sectional view of the first preferred embodiment during an injection stroke;

FIG. 8 is a sectional view of the first preferred embodiment after completion of the injection stroke;

FIG. 9 is a sectional view of the first preferred embodiment, showing the state of disengagement of a seat member from a grip member;

FIG. 10 is a sectional view of the first preferred embodiment, showing that a needle cannula is fully retracted into a barrel;

FIG. 11 is a sectional view of the second preferred embodiment of a disposable syringe according to this invention;

FIG. 12 is a sectional view of the third preferred embodiment of a disposable syringe according to this invention in the initial assembled position;

FIG. 13 is a sectional view of the third preferred embodiment during an injection stroke;

FIG. 14 is a sectional view of the third preferred embodiment, showing that a needle cannula is fully retracted into a barrel;

FIGS. 15 to 18 respectively are sectional views of the fourth, fifth, sixth, and seventh preferred embodiments of a disposable syringe according to this invention;

FIG. 19 is a sectional view of the eighth preferred embodiment of a disposable syringe according to this invention in the initial assembled position;

FIG. 20 is a sectional view of the eighth preferred embodiment, showing that a needle cannula is fully retracted into a barrel;

FIG. 21 is a sectional view of the ninth preferred embodiment of a disposable syringe according to this invention;

FIG. 22 is an exploded sectional view of the tenth preferred embodiment of a disposable syringe according to this invention;

FIG. 23 is a sectional view of the tenth preferred embodiment in the initial assembled position;

FIGS. 24 and 25 respectively are sectional views of the eleventh and twelfth preferred embodiments of a disposable syringe according to this invention;

FIG. 26 is a sectional view of the thirteenth preferred embodiment of a disposable syringe according to this invention in the initial assembled position;

FIG. 27 is a sectional view of the thirteenth preferred embodiment, showing that a needle cannula is fully retracted into a barrel; and

FIG. 28 is a sectional view of the fourteenth preferred embodiment of a disposable syringe according to this invention.

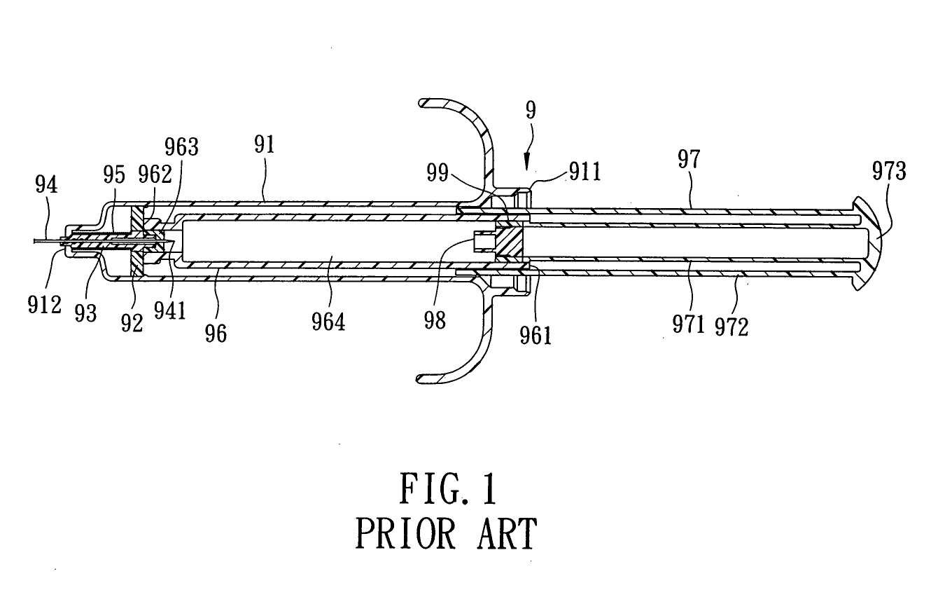

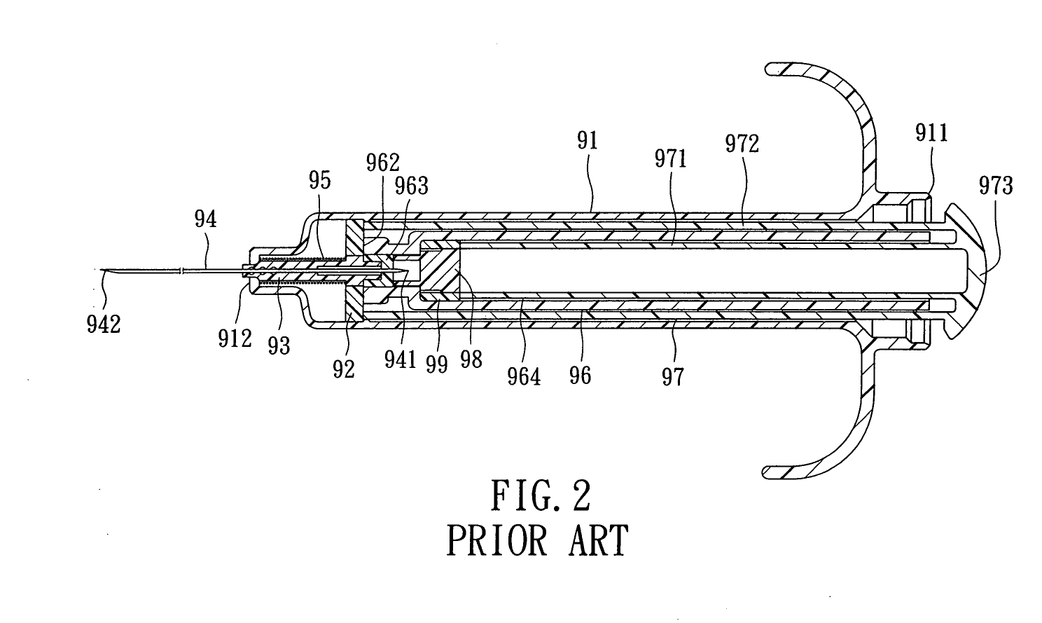

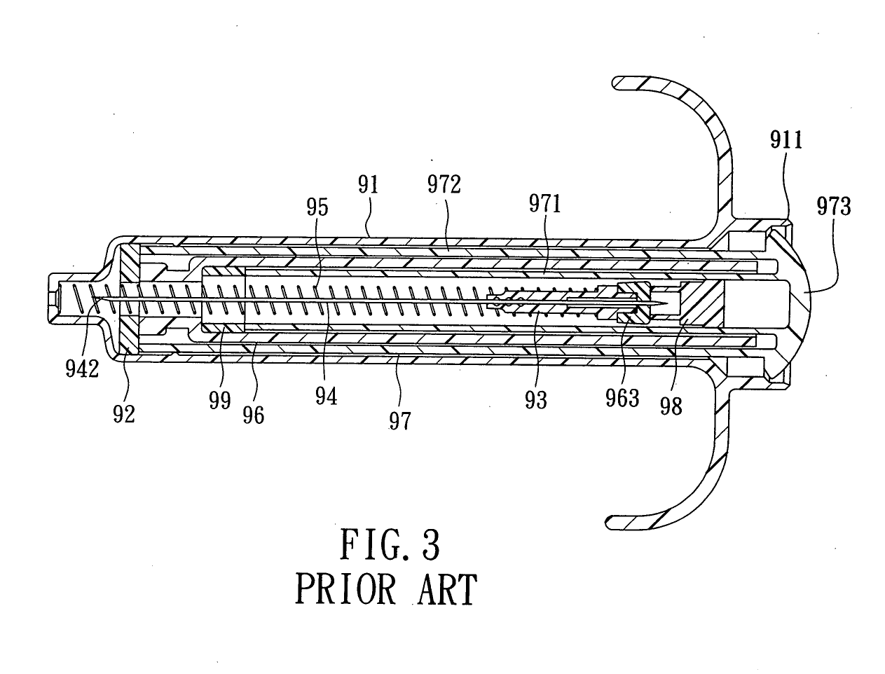

### DETAILED DESCRIPTION OF THE PREFERRED EMBODIMENTS

Before the present invention is described in greater detail, it should be noted that same reference numerals have been used to denote like elements throughout the specification.

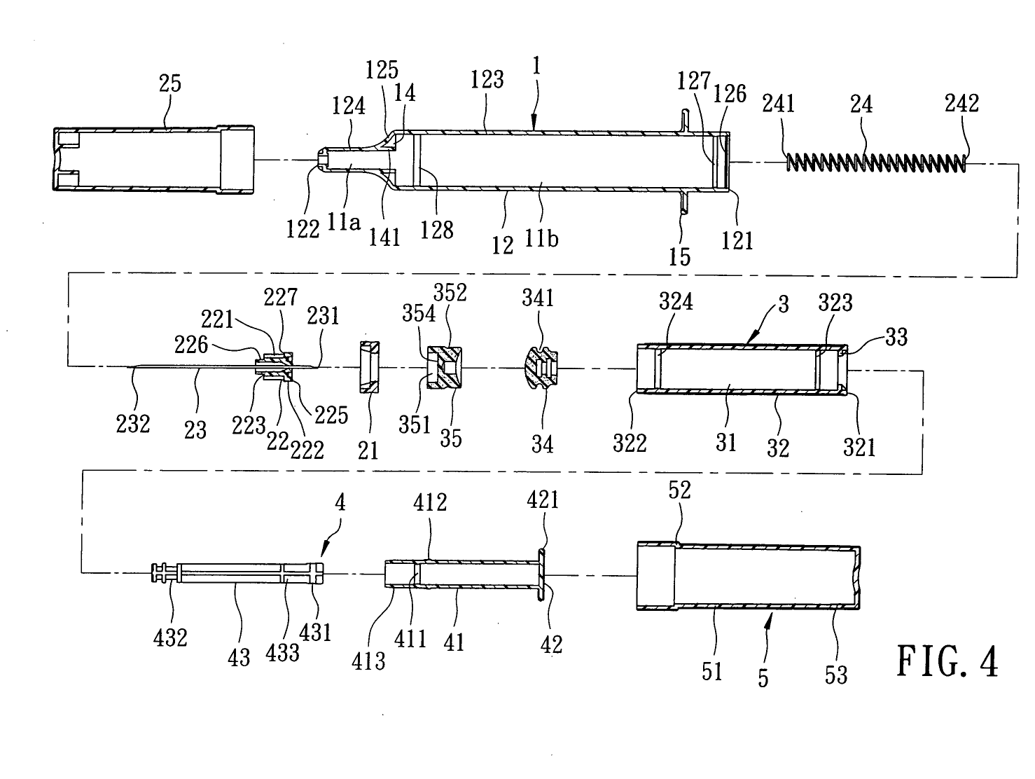

Referring to FIGS. 4 to 6, the first preferred embodiment of a disposable syringe according to the present invention is shown to comprise a barrel 1, a needle cannula 23, a seat member 22, a grip member 21, a biasing member 24, a tip protector 25, a carpule 3, a plunger 4, and a plunger protector 5.

The barrel 1 includes front and rear barrel ends 122, 121 opposite to each other along an axis in an axial direction, a surrounding barrel wall 12 which interconnects the front and rear barrel ends 122, 121, and which includes front and rear wall portions 124, 123 that are disposed proximate to the front and rear barrel ends 122, 121 respectively, and that define front and rear compartments (11a,11b), respectively, and a finger flange 15 which is disposed proximate to the rear barrel end 121. In this embodiment, the rear compartment (11b) is larger than the front compartment (11a) in diameter so as to form a front annular shoulder 125 therebetween. The shoulder 125 has a plurality of fins 14 which extend towards the axis to terminate at an inner peripheral edge 141 that surrounds the axis. Moreover, a recess-like retaining portion 126 is disposed in the rear compartment (11b) adjacent to the rear barrel end 121. An annular stopper 127 is disposed forwardly of the retaining portion 126. A retaining ring 128 is disposed on the rear wall portion 123 adjacent to the front wall portion 124.

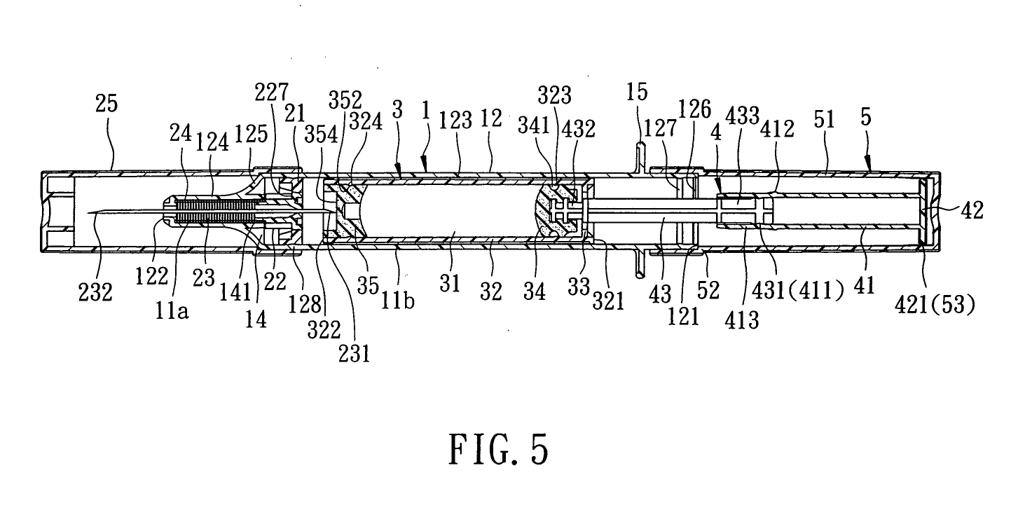

The needle cannula 23 has a tip end 232 which is disposed forwardly of the front barrel end 122 in a position of use (as shown in FIG. 5), and a communicating end 231 which is opposite to the tip end 232 along the axis and which is pointed.

The seat member 22 has a rear seat end surface 225 which holds the needle cannula 23, which permits rearward extension of the communicating end 231 of the needle cannula 23 therethrough, and which confronts rearwardly, a surrounding gripped portion 222 which surrounds the axis, and a surrounding front abutment surface 223 which is opposite to the rear seat end surface 225 and which confronts forwardly. The seat member 22 further has a tubular mount 226 which extends from the surrounding front abutment surface 223 into the front compartment (11a), and which is spaced apart from the inner peripheral edge 141 in radial directions by an inserted clearance that receives a lower end 242 of the biasing member 24 so as to permit the biasing member 24 to abut against the surrounding front abutment surface 223. The biasing member 24 is disposed in the front compartment (11a), and has an upper end 241 abutting against the front barrel end 122. The seat member 22 further has an annular stepped surface 227 which is spaced apart from the fins 14 of the shoulder 125 in the axial direction, and which extends radially and outwardly to join the gripped portion 222, and a plurality of fin spacers 221 which are angularly displaced from one another about the axis, and each of which is interposed between the annular stepped surface 227 and the fins 14 so as to brace the annular stepped surface 227 against the shoulder 125.

The grip member 21 is detachably retained on the rear wall portion 123 by a retaining ring 128, and is configured to be frictionally engaged with the gripped portion 222 so as to provide a resisting force that guards the gripped portion 222 against movement relative to the rear wall portion 123 in the axial direction during a piercing action of the needle cannula 23 for injection, and that permits disengagement of the grip member 21 from the gripped portion 222 so as to enable a subsequent movement of the seat member 22 when the grip member 21 is subjected to a pressing force. The tip protector 25 is removably sleeved on the barrel wall 12 for shielding the needle cannula 23 in the position of use.

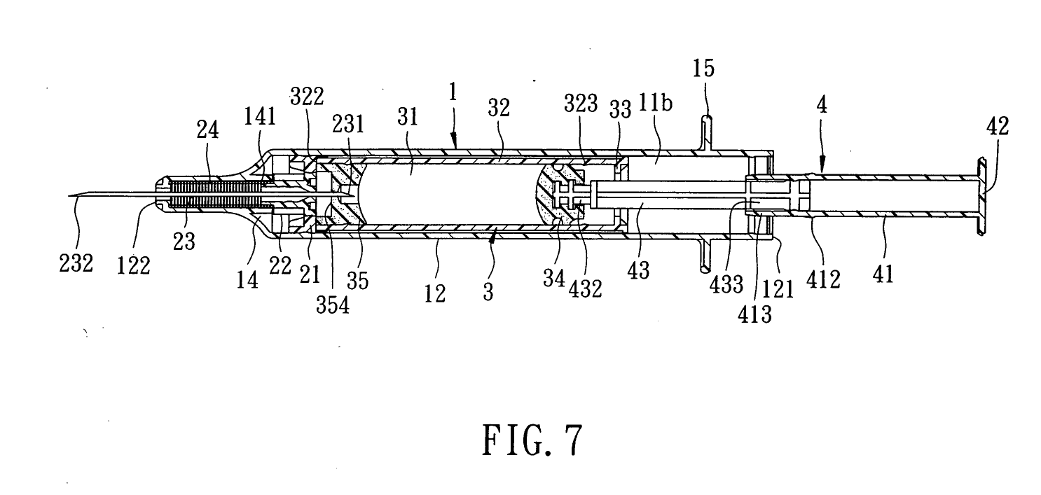

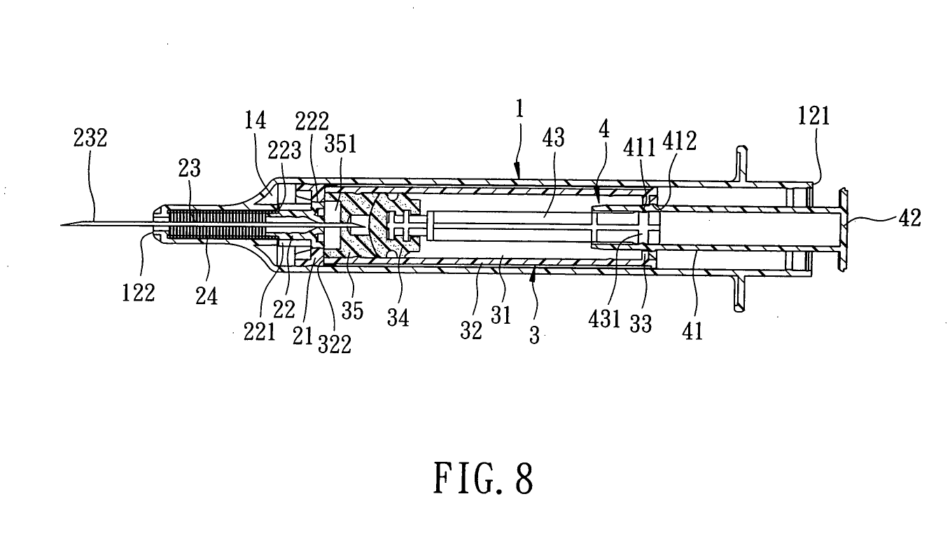

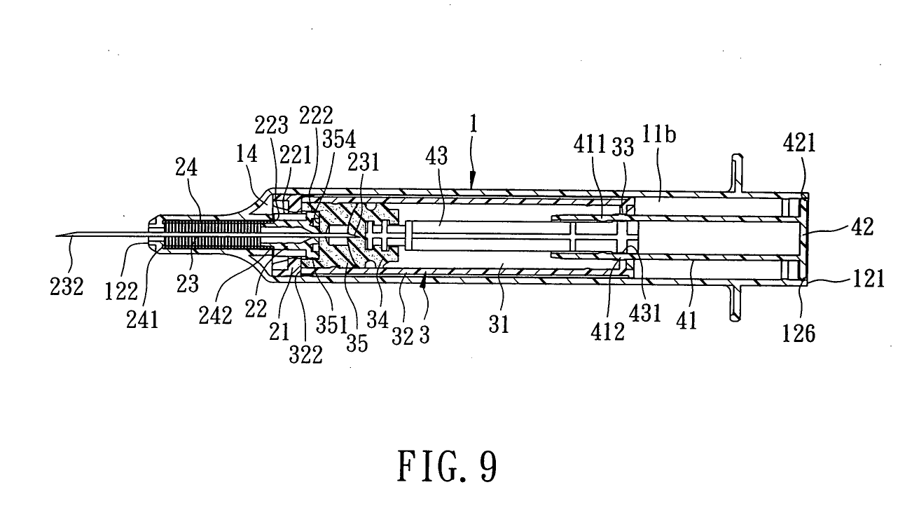

The carpule 3 has a tubular wall 32 which is configured to be fitted in the rear compartment (11b), and which extends in the axial direction to terminate at a tubular edge surface 322 and a rear wall end 321. The carpule 3 further has front and rear seal members 35, 34 which are spaced apart from each other in the axial direction, and which, in cooperation with the tubular wall 32, form a sealed chamber 31 for holding liquid medicament. The front seal member 35 is secured to the tubular wall 32 by means of inter-engagement of female and male engaging portions 352, 324. The rear seal member 34 is limited to the tubular wall 32 by means of inter-engagement of female and male engaging portions 341, 323 so as to guard against undesired movement of the rear seal member 34 before injection, and is forced forwardly to permit disengagement of the female engaging portion 341 from the male engaging portion 323 to be moved to abut against the front seal member 35 in an initial course so as to expel the liquid medicament out of the sealed chamber 31, thereby placing the carpule 3 in a used-up position, as shown in FIG. 8. The front seal member 35 has a mating surface 354 which is configured to have a cavity 351 that extends inwardly and towards the sealed chamber 31. Thus, when the carpule 3 is assembled in the rear compartment (11b), the pointed communicating end 231 of the needle cannula 23 can pierce the mating surface 354 of the front seal member 35 to be retained in and to pass through the mating surface 354 such that the needle cannula 23 is in fluid communication with the sealed chamber 31, as shown in FIG. 7. Moreover, the tubular wall 32 of the carpule 3 has a rear annular shoulder 33 which extends radially and inwardly from the rear wall end 321 to guard against a rearward movement of the rear seal member 34 relative to the tubular wall 32. Thus, once the rear seal member 34 abuts against the front seal member 35 in the used-up position, the pressing force is transmitted through the tubular wall 32 to force the tubular edge surface 322 against the grip member 21 in a subsequent course so as to effect disengagement of the grip member 21 from the gripped portion 222, thereby placing the seat member 22 in a disengaging position, as shown in FIG. 9, where the mating surface 354 is closer to the front barrel end 122 than when the carpule 3 is in the used-up position. At this time, the gripped portion 222 is received in the cavity 351 to ensure that the carpule 3 is kept together with the seat member 22.

The plunger 4 includes first and second plunger bodies 43, 41. The first plunger body 43 has a push end 432 which is connected to the rear seal member 34 by virtue of a frictional force, and a first rear end portion 433 opposite to the push end 432 in the axial direction. The second plunger body 41 includes an operating end 42 which is disposed to extend outwardly of the rear barrel end 121 to be subjected to an external force (i.e., a pressing force applied by a user) that is greater in a subsequent course (i.e., a needle retraction stroke) than in an initial course (i.e., an injection stroke), and a second front end portion 413 which is opposite to the operating end 42, and which is releasably retained with the first rear end portion 433 by means of a mating engagement between an annular retaining protrusion 411 and an annular retaining groove 431 so as to generate a frictional force that is greater than the external force in the initial course and is smaller than the external force in the subsequent course. Moreover, the second plunger body 41 has an abutment 412 which is disposed on the second front end portion 413.

The plunger protector 5 is detachably sleeved on the rear wall portion 123 for shielding the plunger 4, and has a releasably engaging region 53 which is disposed on a peripheral wall 51 thereof and which is brought into frictional engagement with a periphery 421 of the operating end 42 of the plunger 4 when the plunger protector 5 is sleeved on the rear wall portion 123. The plunger protector 5 further has a step edge 52 which is disposed to abut against the rear barrel end 121 for positioning the plunger protector 5 in place.

Referring to FIGS. 5 and 7, in the injection stroke, the tip and plunger protectors 25, 5 are removed first to expose the needle cannula 23 and the plunger 4. The operator holds the finger flange 15 with his/her index and middle fingers and presses the operating end 42 of the plunger 4 forwardly with his/her thumb such that the carpule 3 is moved forwardly in the rear compartment (11b) to cause the communicating end 231 of the needle cannula 23 to pierce the mating surface 354 of the front seal member 35 so as to communicate the needle cannula 23 with the sealed chamber 31. The external pressing force is subsequently applied to the operating end 42 to cause the rear seal member 34 to be moved forwardly along the tubular wall 32 of the carpule 3 so as to place the carpule 3 in the used-up position, thereby completing the injection stroke, as shown in FIG. 8. When the carpule 3 is in the used-up position, the abutment 412 of the plunger 4 abuts against the rear annular shoulder 33 so as to impart the external force to the tubular wall 32 of the carpule 3.

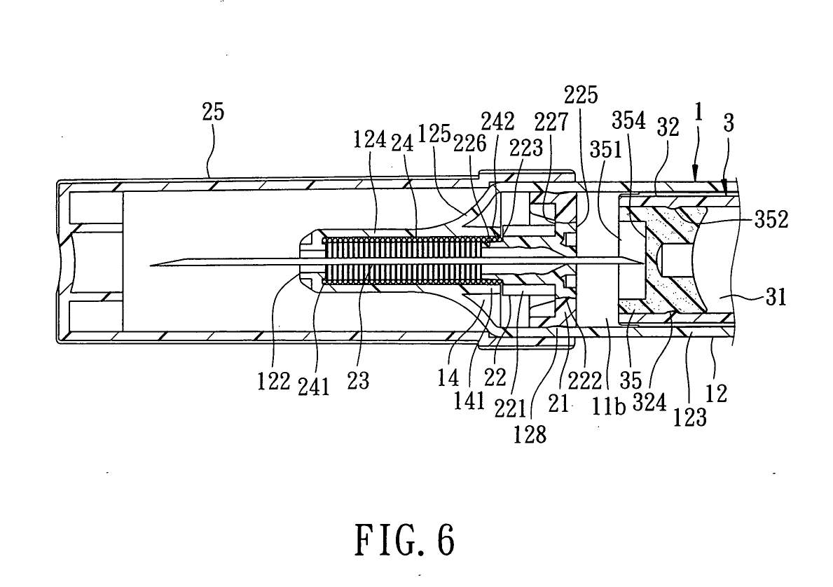

As shown in FIGS. 8 and 9, the external pressing force is subsequently applied to the operating end 42 to force the tubular edge surface 322 against the grip member 21 so as to effect disengagement of the grip member 21 from the gripped portion 222 resulting from blocking of the seat member 22 by the fins 14, thereby placing the seat member 22 in the disengaging position. In this embodiment, referring once again to FIG. 6, the front abutment surface 223 of the seat member 22 is spaced apart from the fins 14 by a small clearance in the position of use. The clearance can serve as a triggering space for facilitating a rearward movement of the seat member 22 after the seat member 22 is moved forwardly with the front seal member 35 to bring the front abutment surface 223 into abutment against the fins 14. Once the grip member 21 is blocked by the fins 14, the external pressing force causes the gripped portion 222 of the seat member 22 to be received in the cavity 351 in the front seal member 35, further causes the disengagement of the retaining protrusion 411 of the second plunger body 41 from the retaining groove 431 in the first plunger body 43 to permit a forward movement of the second plunger body 41 relative to the first plunger body 43 until the periphery 421 of the operating end 42 is retained with the retaining portion 126 of the barrel 1 so as to guard against a rearward movement of the second plunger body 41 for preventing reuse of the syringe.

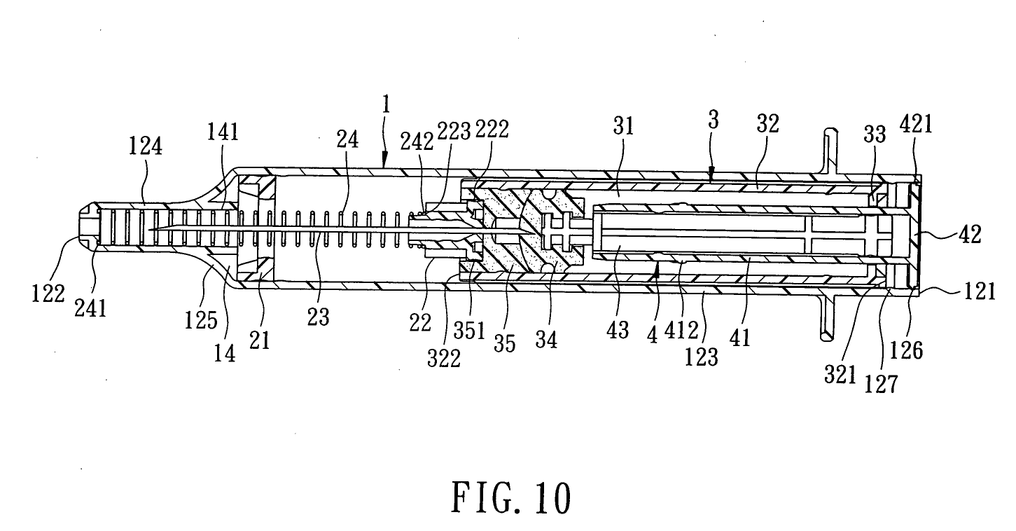

As shown in FIG. 10, when the seat member 22 is in the disengaging position, the biasing force of the biasing member 24 is released to bias the seat member 22 together with the carpule 3 towards the rear barrel end 121 so as to retract the tip end 232 of the needle cannula 23 into the front compartment (11a), thereby placing the needle cannula 23 in a disposal position. The carpule 3 is stopped by the annular stopper 127. At the same time, a large part of the first plunger body 43 is retracted into the second plunger body 41.

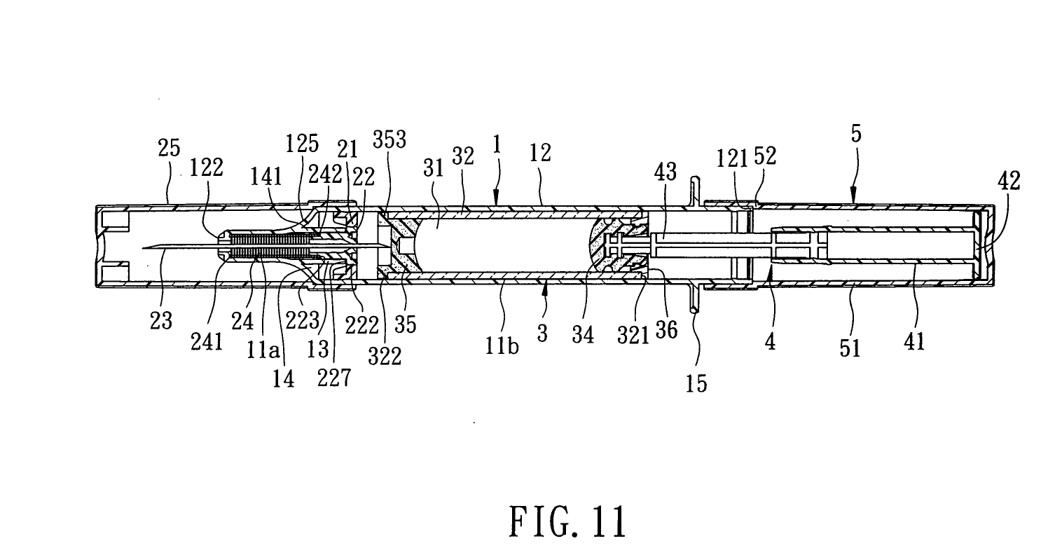

Referring to FIG. 11, the second preferred embodiment of a disposable syringe according to this invention is shown to be similar to the previous embodiment in construction. In the second embodiment, the front seal member 35 has an annular flange 353 which is disposed forwardly of the tubular wall 32 and which is brought to abut against the tubular edge surface 322 so as to ensure fluid-tight engagement of the front seal member 35 with the tubular wall 32. An annular barrier 36 is disposed to abut against the rear wall end 321 of the carpule 3, and is detachably sleeved on the rear seal member 34 to serve as the rear annular shoulder. Moreover, a sleeve 13 extends rearwardly from the inner peripheral edge 141 of the front annular shoulder 125 such that the annular stepped surface 227 of the seat member 22 abuts against the sleeve 13 in the position of use.

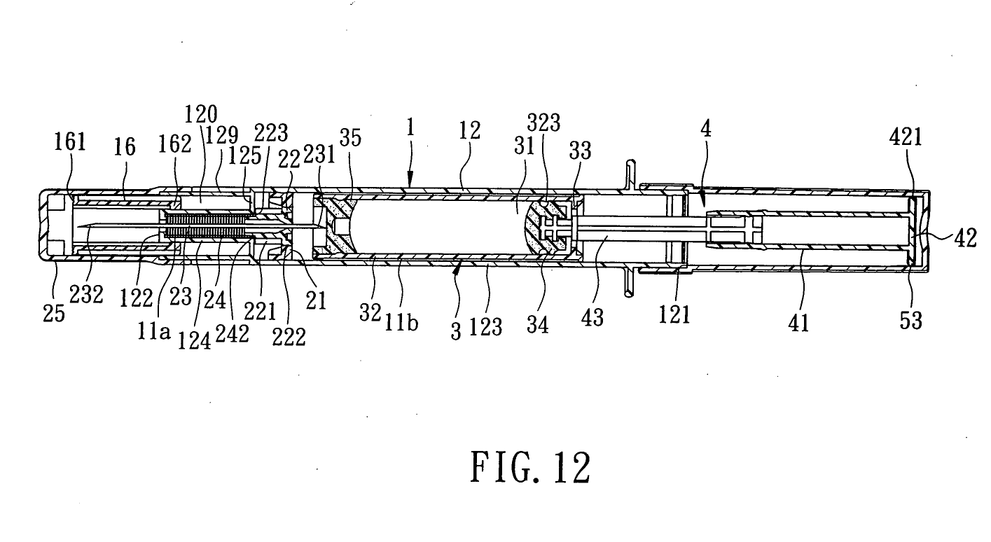

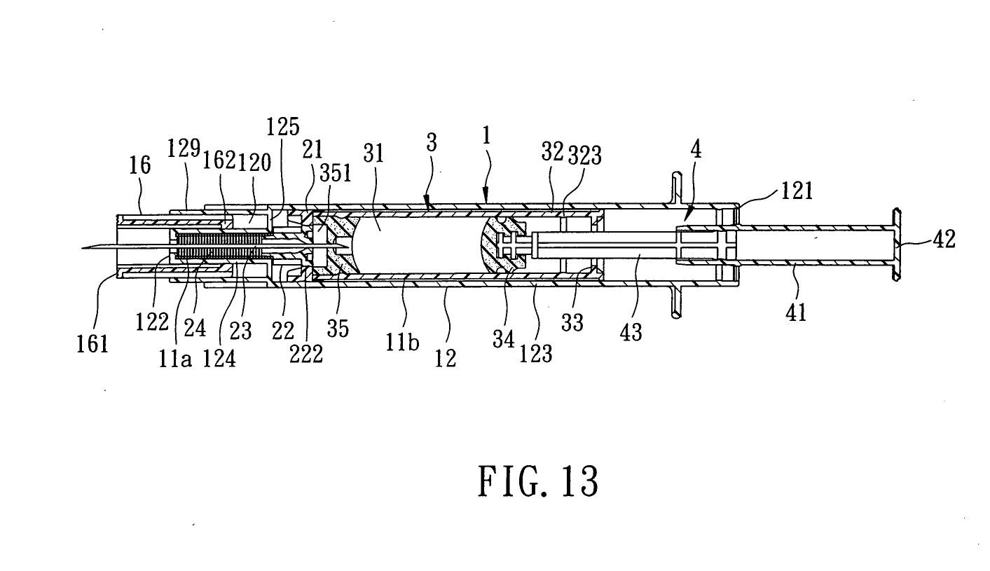

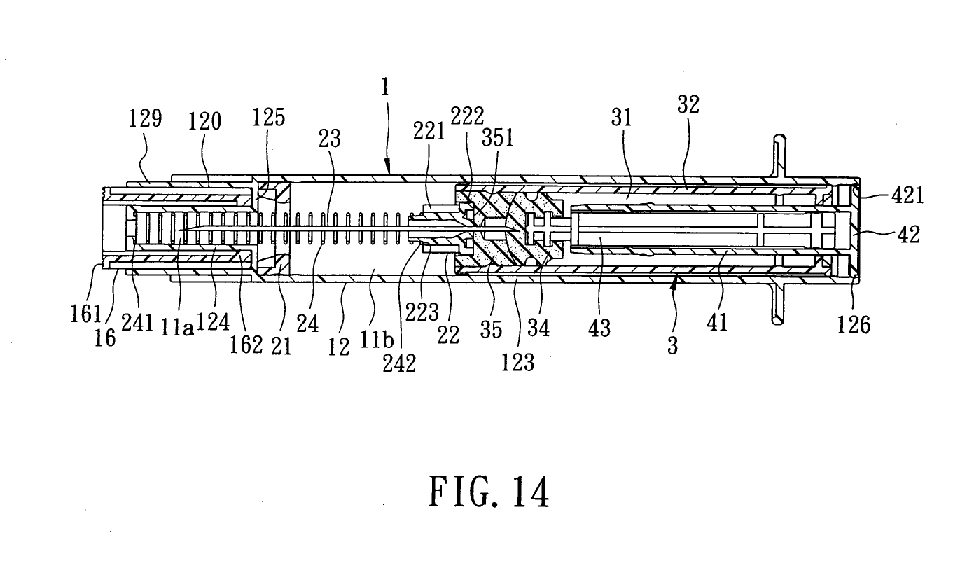

Referring to FIGS. 12 to 14, the third preferred embodiment of a disposable syringe according to this invention is shown to be similar to the first embodiment in construction. In the third embodiment, the barrel 1 has a tubular front wall 129 which extends forwardly from the rear wall portion 123 in the axial direction and which is spaced apart from the front wall portion 124 in radial directions to define a sliding channel 120 therebetween. A tubular touching member 16 is received in the sliding channel 120, and includes a tubular touching wall which surrounds the axis, and which has a rear engaging end 162 that is slidably engaged with the front wall portion 124, and a front touching end 161 that is movable by virtue of a sliding movement of the rear engaging end 162 between a forward position, where the touching end 161 is disposed forwardly of the tip end 232 of the needle cannula 23 for touching a patient's skin prior to piercing of the skin with the tip end 232, and a rearward position, where the touching end 161 is retracted to expose the tip end 232 for a piercing action. The front touching end 161 has a rough surface. The needle protector 25 removably covers the tubular front wall 129 so as to shield the needle cannula 23 before use.

During the injection stroke, the front touching end 161 is brought to touch a patient's skin and is forced to move rearwardly to the rearward position to expose the tip end 232 of the needle cannula 23 for the piercing action. When the touching member 16 comes into contact with the patient's skin, the patient's attention to the needle cannula 23 is diverted thereto, thereby minimizing the injection pain.

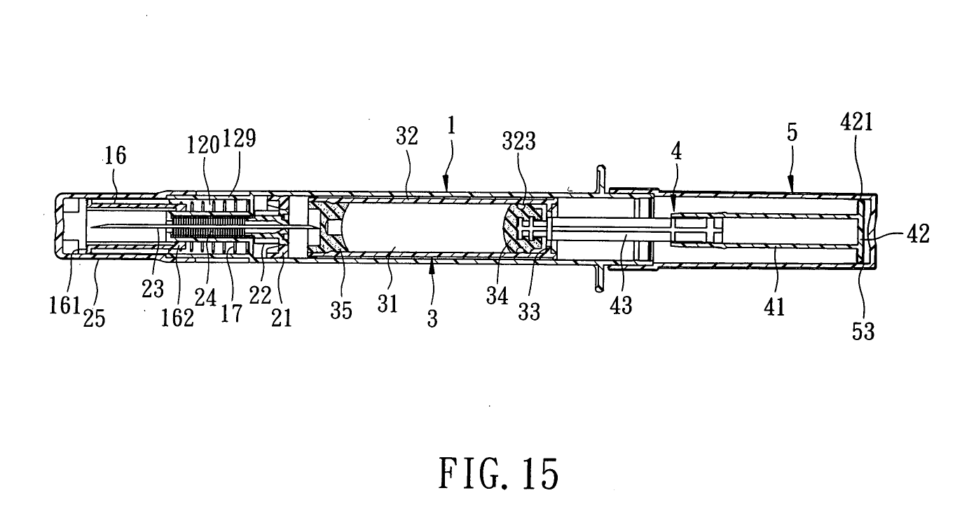

Referring to FIG. 15, the fourth preferred embodiment of a disposable syringe according to this invention is shown to be similar to the third embodiment in construction. In the fourth embodiment, the syringe further comprises a spring 17 which is disposed in the sliding channel 120 to bias the touching end 161 of the touching member 16 to the forward position.

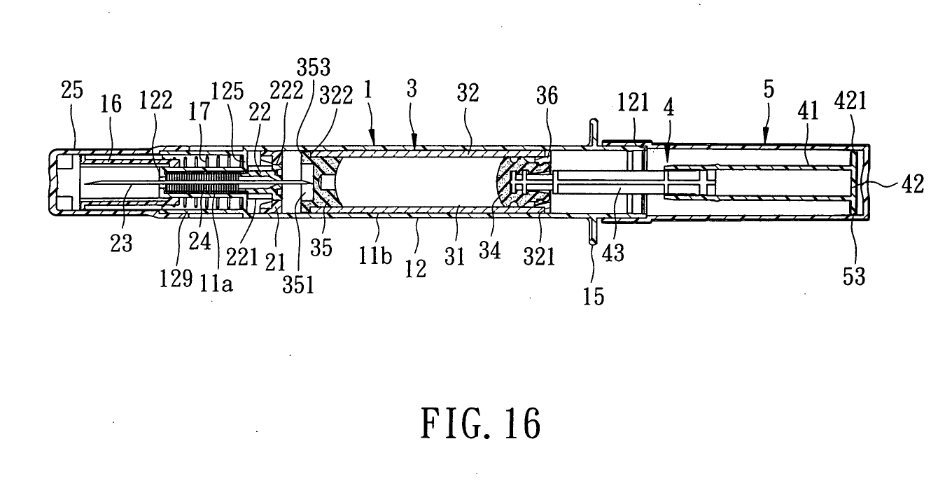

Referring to FIG. 16, the fifth preferred embodiment of a disposable syringe according to this invention is shown to be similar to the fourth embodiment in construction. In the fifth embodiment, the front seal member 35 has an annular flange 353 which is disposed forwardly of the tubular wall 32 and which is brought to abut against the tubular edge surface 322 so as to ensure fluid-tight engagement between the front seal member 35 and the tubular wall 32. An annular barrier 36 is disposed to abut against the rear wall end 321 of the carpule 3, and is detachably sleeved on the rear seal member 34 to serve as the rear annular shoulder.

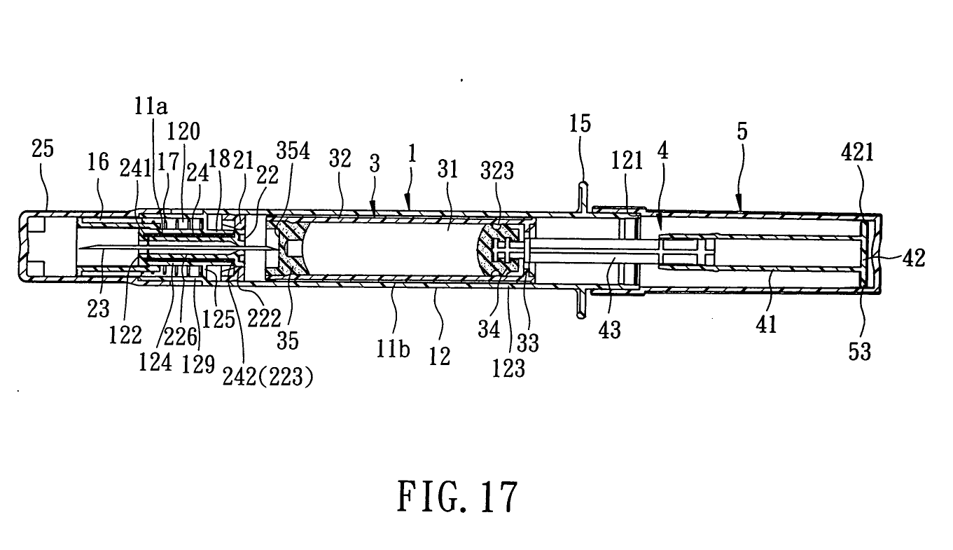

Referring to FIG. 17, the sixth preferred embodiment of a disposable syringe according to this invention is shown to be similar to the fourth embodiment in construction. In the sixth embodiment, the tubular mount 226 of the seat member 22 extends through the front compartment (11a) to abut against the front barrel end 122. The barrel 1 has a tubular spacer 18 which is integrally formed with and which extends rearwardly from the front annular shoulder 125 to permit abutment of the front abutment surface 223 of the seat member 22 there against, and which encloses the lower end 242 of the biasing member 24 for steadying the biasing action of the biasing member 24.

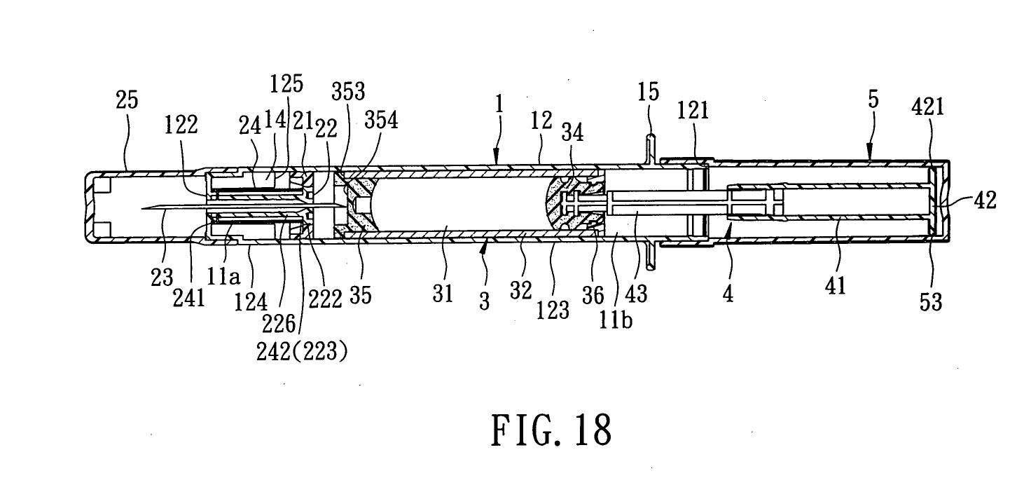

Referring to FIG. 18, the seventh preferred embodiment of a disposable syringe according to this invention is shown to be similar to the sixth embodiment in construction. In the seventh embodiment, the carpule 3 is the same as that of the second embodiment. The surrounding front abutment surface 223 of the seat member 22 is disposed to be spaced apart from the fins 14 of the front annular shoulder 125 so as to expose the lower end 242 of the biasing member 24. Thus, the tubular spacer 18 is omitted, and the biasing member 24 may have a larger dimension to enhance the biasing strength thereof.

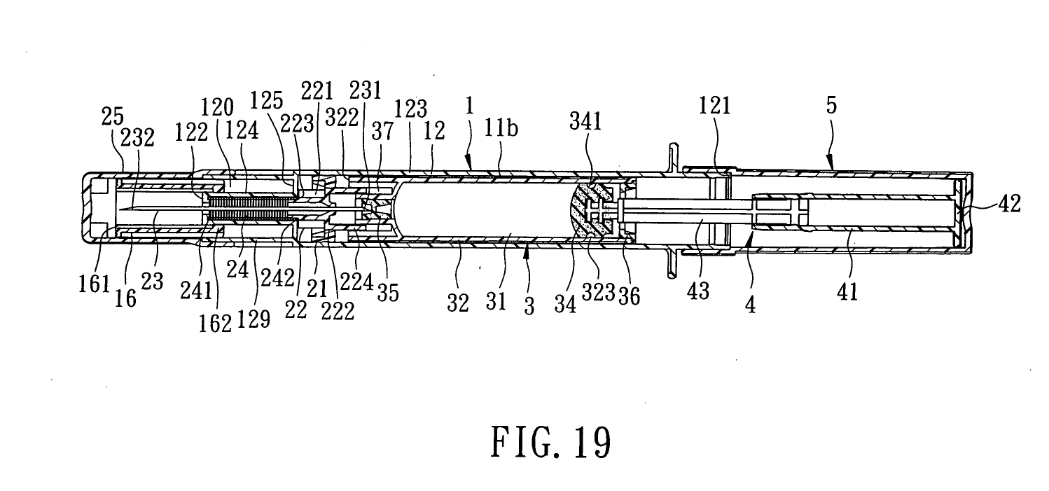

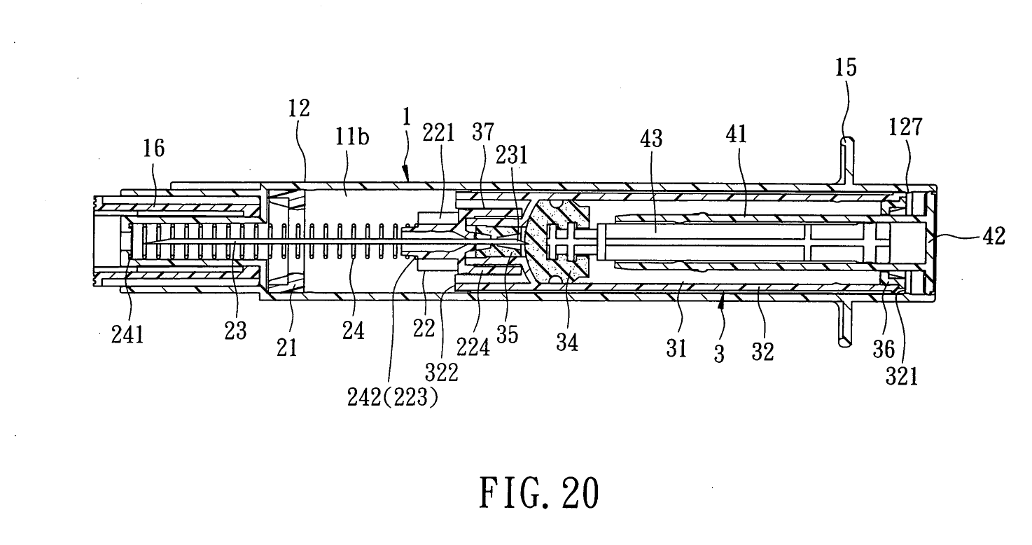

Referring to FIGS. 19 and 20, the eighth preferred embodiment of a disposable syringe according to this invention is shown to be similar to the third embodiment in construction. In the eighth embodiment, the carpule 3 has an annular key slot 37 which is disposed adjacent to the tubular edge surface 322 and which extends in the axial direction to surround the front seal member 35. The seat member 22 has an annular key 224 which extends rearwardly from the gripped portion 222 and which is disposed to slide in and along the key slot 37 for facilitating a forward movement of the carpule 3 along the axis when the seat member 22 is brought to the disengaging position. Besides, the engagement of the key slot 37 with the key 224 facilitates rearward movement of the seat member 22 together with the carpule 3 toward the rear barrel end 121 so as to ensure retraction of the needle cannula 23 to the disposal position. By means of the provision of the key slot 37 and the key 224, the carpule 3 can be firmly received in the rear compartment (11b), thereby preventing the communicating end 231 from accidentally piercing the front seal member 35 during transport.

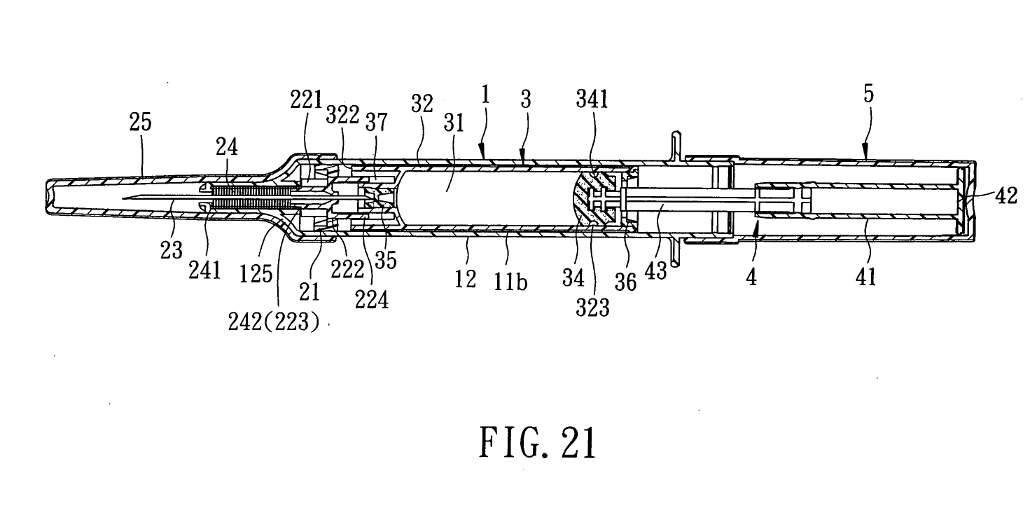

Referring to FIG. 21, the ninth preferred embodiment of a disposable syringe according to this invention is shown to be similar to the eighth embodiment in construction, except that the barrel 1 is the same as that of the first embodiment.

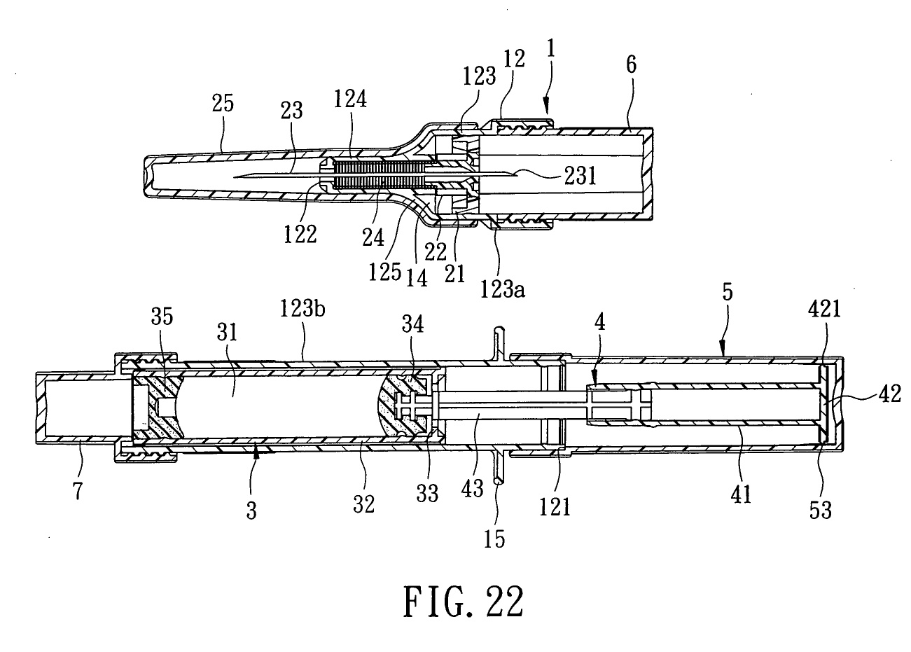

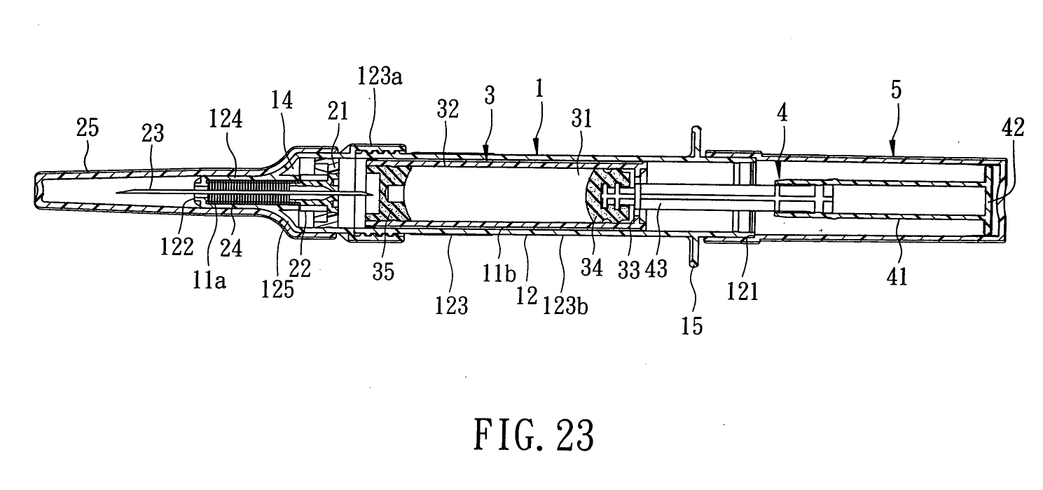

Referring to FIGS. 22 and 23, the tenth preferred embodiment of a disposable syringe according to this invention is shown to be similar to the first embodiment in construction. In the tenth embodiment, the barrel 1 has two parts. Particularly, the rear wall portion 123 of the barrel wall 12 includes a first wall segment (123a) which is integrally formed with and which extends rearwardly from the front wall portion 124 to receive the seat member 22 and the grip member 21 as a first unit, and a second wall segment (123b) which is threadedly engaged with the first wall segment (123a) to receive the carpule 3 and the plunger 4 as a second unit. A first end cap 6 is threadedly connected to the first wall segment (123a) for shielding the communicating end 231 of the needle cannula 23. A second end cap 7 is threadedly connected to the second wall segment (123b) for shielding the carpule 3. Thus, the first and second units can be packed separately to meet a variety of injection requirements.

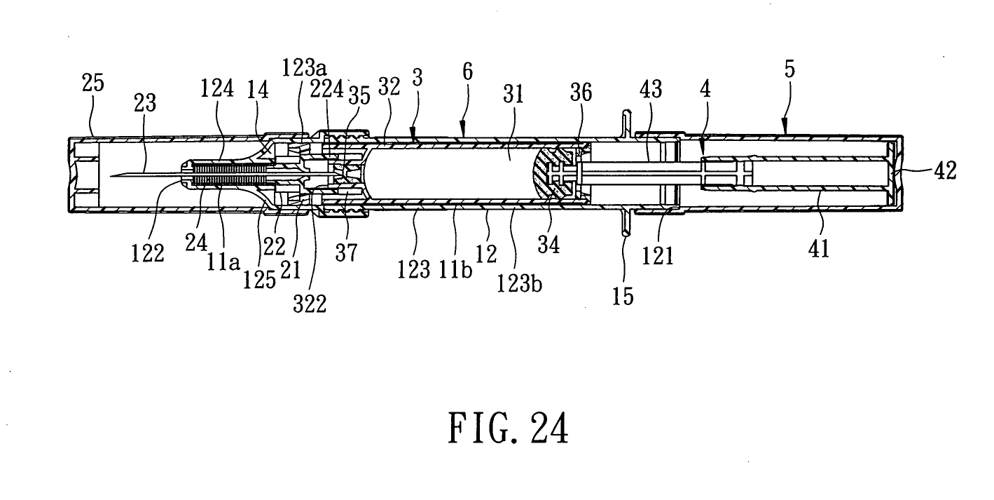

Referring to FIG. 24, the eleventh preferred embodiment of a disposable syringe according to this invention is shown to be similar to the tenth embodiment in construction, except that the seat member 22 and the carpule 3 are the same as those of the ninth embodiment.

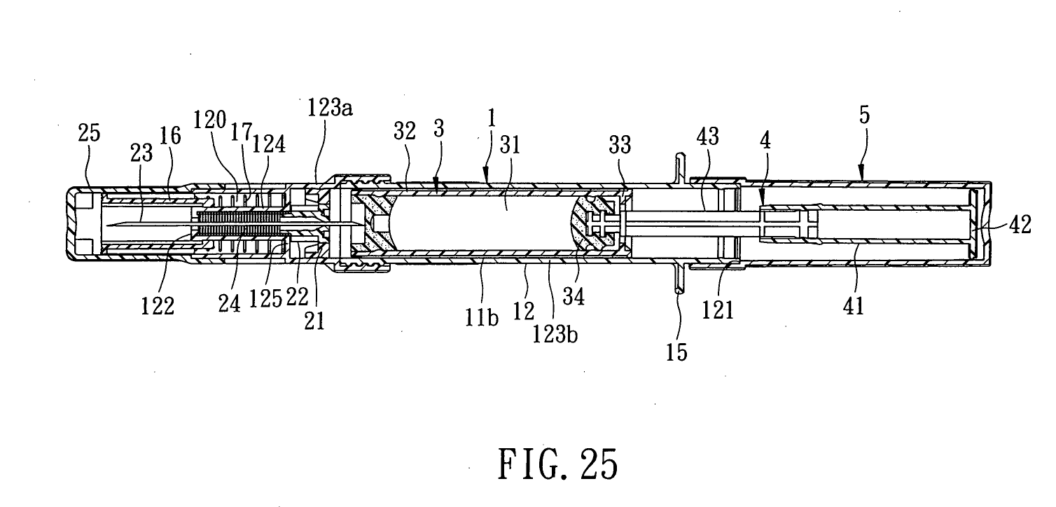

Referring to FIG. 25, the twelfth preferred embodiment of a disposable syringe according to this invention is shown to be similar to the tenth embodiment in construction, except that the front wall portion 124 of the barrel 1, the tubular touching member 16, and the spring 17 are the same as those of the fourth embodiment.

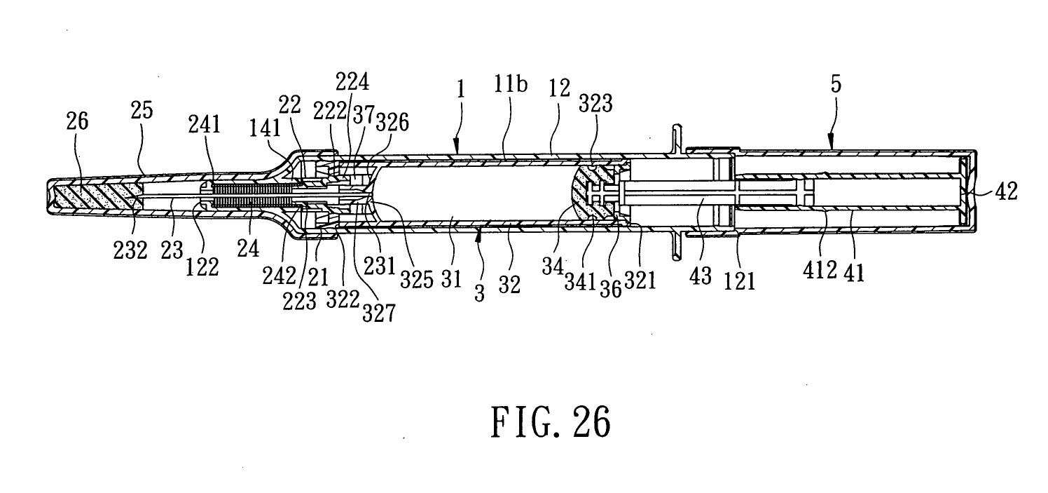

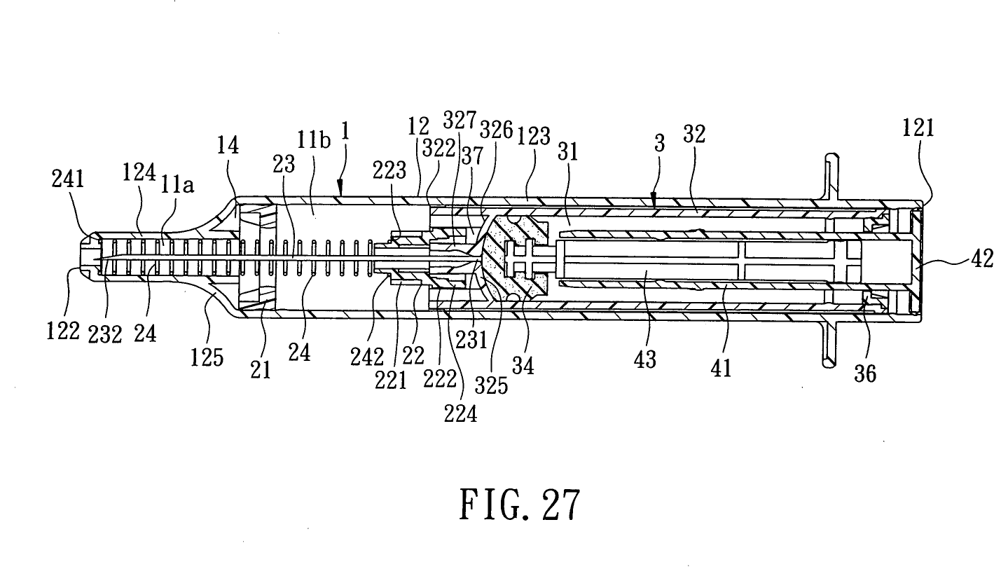

Referring to FIGS. 26 and 27, the thirteenth preferred embodiment of a disposable syringe according to this invention is shown to be similar to the first embodiment in construction. In the thirteenth embodiment, the front seal member 325 of the carpule 3 is integrally formed with the tubular wall 32. Instead of the seat member 22 which holds the needle cannula 23, the carpule 3 has a tubular needle holding portion 327 which extends forwardly from the mating surface 326 along the axis and which holds the communicating end 231 of the needle cannula 23 to communicate the needle cannula 23 with the sealed chamber 31. The carpule 3 further has an annular key slot 37 which extends forwardly from the mating surface 326 in the axial direction to surround the needle holding portion 327. The seat member 22 has an annular key 224 which extends rearwardly from the gripped portion 222 and which is disposed to slide in and along the key slot 37. Moreover, an elastomeric packing 26 is stuffed in the tip protector 25 such that when the tip protector 25 is sleeved on the barrel wall 12, the tip end 232 of the needle cannula 23 is trapped in the elastomeric packing 26, thereby preventing fluid leakage.

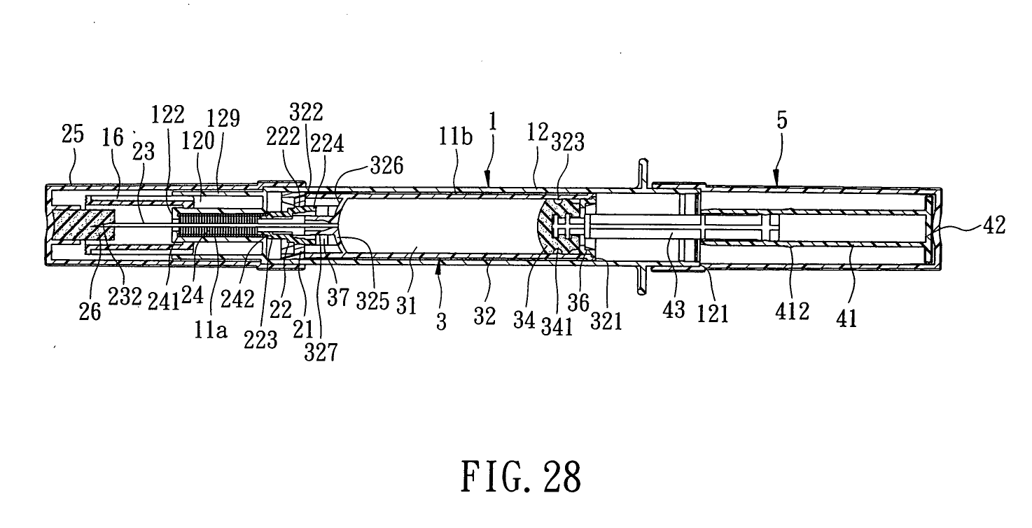

Referring to FIG. 28, the fourteenth preferred embodiment of a disposable syringe according to this invention is shown to be similar to the thirteenth embodiment in construction, except that the barrel 1 and the tubular touching member 16 are the same as those of the third embodiment.

As illustrated, the disposable syringe of this invention has the following advantages:

1. Since the communicating end 231 of the needle cannula 23 is held by the carpule 3 after the carpule 3 is mounted in the barrel 1, and since the seat member 22 is mated with the front seal member 35, 325 when the seat member 22 is in the disengaging position, the seat member 22 can be moved together with the carpule 3 towards the rear barrel end 121 by the biasing action of the biasing member 24 in the needle retraction stroke, thereby ensuring the retraction of the needle cannula 23 into the barrel 1.

2. Since the carpule 3 can be pre-assembled in the barrel 1, clinical injection operation by health care workers is simplified and convenient to conduct. In addition, the syringe can be conveniently rendered unreusable after injection in a single operation.

3. Since, the resisting force generated between the grip member 21 and the seat member 22, and the frictional force generated between the first and second plunger bodies 43, 41 during pressing of the plunger 4 are overcome by the external pressing force, the injection and needle retraction strokes are smooth and successful.

4. Since the operating end 42 of the plunger 4 is retained with the retaining portion 126 of the barrel 1 to guard against a rearward movement of the plunger 4 after the operating end 42 is fully pressed in the barrel 1, reuse of the syringe can be prevented.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

We claim:

1. A disposable syringe with a built-in carpule comprising:
a barrel including front and rear barrel ends opposite to each other along an axis in an axial direction, a surrounding barrel wall which interconnects said front and rear barrel ends, and which includes front and rear wall portions that are disposed proximate to said front and rear barrel ends, respectively, and that define front and rear compartments, respectively;
a needle cannula having a tip end which is disposed forwardly of said front barrel end in a position of use, and a communicating end which is opposite to said tip end along the axis;
a seat member which has a surrounding gripped portion surrounding the axis;
a grip member which is detachably retained on said rear wall portion, and which is configured to be frictionally engaged with said gripped portion to provide a resisting force that guards said gripped portion against movement relative to said rear wall portion in the axial direction during a piercing action of said needle cannula for injection, and that permits disengagement of said grip member from said gripped portion so as to enable a subsequent movement of said seat member when said grip member is subjected to a pressing force;
a carpule which has a tubular wall that is configured to be fitted in said rear compartment, and that extends towards said grip member to terminate at a tubular edge surface, and front and rear seal members that are spaced apart from each other in the axial direction, and that, in cooperation with said tubular wall, form a sealed chamber for holding liquid medicament, said rear seal member being disposed to be movable to abut against said front seal member in an initial course so as to expel the liquid medicament out of said sealed chamber, thereby placing said carpule in a used-up position, said front seal member having a mating surface which is configured to permit said communicating end to be retained with and to pass through said mating surface such that said needle cannula is in fluid communication with said sealed chamber, and such that once said rear seal member abuts against said front seal member in the used-up position, the pressing force is transmitted through said tubular wall to force said tubular edge surface against said grip member in a subsequent course so as to effect disengagement of said grip member from said gripped portion, thereby placing said seat member in a disengaging position, where said mating surface is closer to said front barrel end than that in the used-up position;
a plunger which has a push end that is disposed to engage and move said rear seal member forwardly along said tubular wall to bring said carpule to the used-up position, and to move said mating surface forwardly from the used-up position so as to permit said seat member to be placed in the disengaging position, and an operating end that is opposite to said push end in the axial direction, and that is disposed to extend outwardly of said rear barrel end to be subjected to an external force which is greater in the subsequent course than in the initial course; and
a biasing member which is disposed in said front compartment such that, once said seat member is in the disengaging position, said biasing member biases said seat member together with said carpule towards said rear barrel end to retract said tip end of said needle cannula into said barrel, thereby placing said needle cannula in a disposal position.

2. The disposable syringe with a built-in carpule of claim 1, wherein said plunger includes
a first plunger body having said push end which is disposed to engage and move with said rear seal member, and a first rear end portion opposite to said push end, and
a second plunger body including said operating end, and a second front end portion which is opposite to said operating end, and which is releasably retained with said first rear end portion by a frictional force that is greater than the external force in the initial course so as to permit said rear seal member to be moved forwardly by the external force along said tubular wall to thereby place said carpule in the used-up position, and that is smaller than the external force in the subsequent course to permit said second plunger body to move relative to said first plunger body so as to impart the external force to said tubular wall to thereby force said tubular edge surface against said grip member with the pressing force.

3. The disposable syringe with a built-in carpule of claim 2, wherein said tubular wall of said carpule has a rear wall end which is opposite to said tubular edge surface, and a rear annular shoulder which extends radially and inwardly from said rear wall end, said second plunger body having an abutment which is disposed on said second front end portion of said second plunger body and which is configured to abut against said rear annular shoulder when said carpule is in the used-up position so as to impart the external force to said tubular wall.

4. The disposable syringe with a built-in carpule of claim 3, wherein said rear annular shoulder is configured to guard against a rearward movement of said rear seal member relative to said tubular wall.

5. The disposable syringe with a built-in carpule of claim 2, wherein said barrel wall has a retaining portion adjacent to said rear barrel end, said second plunger body being configured such that said operating end is retained with said retaining portion once said seat member is placed in the disengaging position so as to guard against a rearward movement of said second plunger body.

6. The disposable syringe with a built-in carpule of claim 2, wherein said first rear end portion and said second front end portion respectively have an annular retaining groove and an annular retaining protrusion which are matingly engaged with each other to generate the frictional force.

7. The disposable syringe with a built-in carpule of claim 1, wherein said seat member is disposed to fix said needle cannula and to permit rearward extension of said communicating end of said needle cannula therethrough, said communicating end being configured to be pointed so as to pierce said mating surface of said front seal member to thereby communicate said needle cannula with said sealed chamber.

8. The disposable syringe with a built-in carpule of claim 7, wherein said mating surface of said front seal member has a cavity which extends inwardly and towards said sealed chamber so as to receive said gripped portion of said seat member when said seat member is in the disengaging position, thereby ensuring that said carpule is kept together with said seat member when said seat member is biased towards said rear barrel end.

9. The disposable syringe with a built-in carpule of claim 7, wherein said front seal member has an annular flange which is disposed forwardly of said tubular wall and which is brought to abut against said tubular edge surface so as to ensure fluid-tight engagement between said front seal member and said tubular wall.

10. The disposable syringe with a built-in carpule of claim 7, wherein said rear wall portion includes a first wall segment which is integrally formed with and which extends rearwardly from said front wall portion to receive said seat member and said grip member, and a second wall segment which is detachably coupled to said first wall segment to receive said carpule and said plunger.

11. The disposable syringe with a built-in carpule of claim 10, further comprising a first end cap which is connected to said first wall segment for shielding said communicating end of said needle cannula, and a second end cap which is connected to said second wall segment for shielding said carpule.

12. The disposable syringe with a built-in carpule of claim 1, wherein said carpule has a tubular needle holding portion which extends forwardly from said mating surface along the axis and which holds said communicating end of said needle cannula to communicate said needle cannula with said sealed chamber.

13. The disposable syringe with a built-in carpule of claim 12, further comprising a tip protector which is removably sleeved on said barrel wall for shielding said needle cannula, and an elastomeric packing which is stuffed in said tip protector such that when said tip protector is sleeved on said barrel wall, said tip end of said needle cannula is trapped in said elastomeric packing.

14. The disposable syringe with a built-in carpule of claim 1, wherein said rear compartment is larger than said front compartment in diameter so as to form a front annular shoulder therebetween, which extends towards the axis and terminates at an inner peripheral edge that surrounds said needle cannula.

15. The disposable syringe with a built-in carpule of claim 14, wherein said seat member includes a surrounding front abutment surface which is disposed to confront said front compartment, and a rear seat end surface which is opposite to said surrounding front abutment surface, and which confronts said mating surface to hold said communicating end steadily so as to facilitate passage of said communicating end through said mating surface.

16. The disposable syringe with a built-in carpule of claim 15, wherein said seat member includes a tubular mount which extends from said surrounding front abutment surface into said front compartment, and which is spaced apart from said inner peripheral edge in radial directions by an inserted clearance that receives a lower end of said biasing member so as to permit said biasing member to abut against said surrounding front abutment surface.

17. The disposable syringe with a built-in carpule of claim 16, wherein said tubular mount extends through said front compartment to engage with said front barrel end.

18. The disposable syringe with a built-in carpule of claim 17, wherein said surrounding front abutment surface is disposed to be spaced apart from said front annular shoulder such that said lower end of said biasing member is exposed.

19. The disposable syringe with a built-in carpule of claim 18, wherein said barrel has a tubular spacer which is disposed between said front annular shoulder and said surrounding front abutment surface of said seat member, and which encloses said exposed lower end of said biasing member, said tubular spacer being configured to be integrally formed with said front annular shoulder.

20. The disposable syringe with a built-in carpule of claim 16, wherein said seat member includes an annular stepped surface which is spaced apart from said front annular shoulder in the axial direction, and which extends radially and outwardly to join said surrounding gripped portion, and a plurality of fin spacers which are angularly displaced from one another about the axis, and each of which is interposed between said annular stepped surface and said front annular shoulder so as to brace said annular stepped surface against said front annular shoulder.

21. The disposable syringe with a built-in carpule of claim 1, wherein said carpule has a key slot which is disposed adjacent to said tubular edge surface and which extends in the axial direction, said seat member having a key which extends rearwardly from said gripped portion and which is disposed to slide in and along said key slot.

22. The disposable syringe with a built-in carpule of claim 1, further comprising a tubular touching member that includes a tubular touching wall which surrounds the axis, and which has a rear engaging end that is slidably engaged with said front wall portion, and a front touching end that is movable by virtue of a sliding movement of said rear engaging end between a forward position, where said touching end is disposed forwardly of said tip end of said needle cannula for touching a patient's skin prior to piercing the patient's skin with said tip end, and a rearward position, where said touching end is retracted to expose said tip end for a piercing action.

23. The disposable syringe with a built-in carpule of claim 22, further comprising a spring which is disposed to bias said touching end to the forward position.

24. The disposable syringe with a built-in carpule of claim 22, wherein said barrel has a tubular front wall which extends forwardly from said rear wall portion in the axial direction and which is spaced apart from said front wall portion in radial directions to define a sliding channel therebetween, said sliding channel being configured to receive said touching member and guide the movement of said rear engaging end relative thereto, said syringe further comprising a needle protector which is configured to removably cover said tubular front wall so as to shield said needle cannula.

25. The disposable syringe with a built-in carpule of claim 1, further comprising a plunger protector which is detachably sleeved on said rear wall portion for shielding said plunger, and which has a releasably engaging region that is brought into frictional engagement with said operating end of said plunger when said plunger protector is sleeved on said rear wall portion.
